(19) United States
(12) Patent Application Publication
(10) Pub. No.: US 2004/0208925 A1
(43) Pub. Date: Oct. 21, 2004

(54) Dispersible alendronate microparticle formulation

(75) Inventors: Levent Oner, Cankaya/Ankara (TR); Umit Cifter, Maslak/Istanbul (TR); Nisa Sakarya, Maslak/Istanbul (TR); Ali Turkyilmaz, Maslak/Istanbul (TR)

Correspondence Address: THE FIRM OF KARL F ROSS, 5676 RIVERDALE AVENUE, PO BOX 900, RIVERDALE (BRONX), NY, 10471-0900, US

(73) Assignee: SANOVEL ILAC SANAYI VE TICARET ANONIM SIRKETI

(21) Appl. No.: 10/824,695

(22) Filed: Apr. 14, 2004

(30) Foreign Application Priority Data
Apr. 18, 2003 (TR) 2003/510

(51) Int. Cl. A61K 31/66; A61K 9/20

(52) U.S. Cl. 424/465; 514/102

(57) ABSTRACT

This invention relates to the preparation of the pharmaceutical dosage form that is packaged into sachets, which contain alendronate microparticles coated with polymers resistant to salivary pH to decrease the esophageal and gastric side effects of alendronate, therapeutic amounts of alginic acid or sodium alginate and at least one sweetener or a mixture of sweeteners.

### TECHNICAL FIELD

This invention relates to the pharmaceutical dosage forms that are packaged into sachets, which contain therapeutic amounts of alendronate (alendronate sodium or alendronate monosodium trihydrate) micro-particles that are prevented to be released into saliva, and alginic acid or sodium alginate and at least one sweetener or a mixture of sweeteners, to be administered orally after dispersed in a glass of 250 ml. water.

The above mentioned invention is associated with the decrease of potential side effects of alendronate that may arise in esophagus and stomach. The methods in this invention are:

the use of sodium alginate or alginic acid to prevent oesophageal reflux, ulcer and heartburn that may arise during alendronate use

prevention any irritation related to alendronate sodium during its passage through esophagus by ensuring the release of it in stomach.

### BACKGROUND OF THE INVENTION

Alendronate sodium is chemically described as (4-amino-1-hydroxybutylidene) bisphosphonic acid monosodium salt trihydrate. Patients complain about heartburn after use of alendronate. Alendronate sodium tablets are recommended to be used with a glass of water. In U.S. Pat. No. 5,853,759, it has been disclosed that alendronate tablets taken without a glass of water may cause irritation.

Currently therapies with alendronate may be grouped as permanent treatments with daily doses and treatments to be applied with given intervals (once in three days, once in a week or once in 15 days). It is known that in both treatment forms alendronate causes disorders such as esophageal reflux, heartburn and esophagitis. The patent with PCT application No. of WO 99/04773, relates to alendronate treatment with given intervals such as its use once a week, once in two weeks or once a month to decrease its gastrointestinal side effects. Additionally, the use of the pharmaceutical compound in combination with H2 receptor blockers and/or proton pump inhibitors is disclosed in this patent.

The effervescent alendronate formulations are disclosed in U.S. patent No. of U.S. Pat. No. 5.853.759 and in addition to that, procedures for preparing the liquid alendronate formulations are disclosed in PCT Application No. of WO 98/14196. In these patents, alendronate is in dissolved form, i.e., alendronate that gets in contact with saliva is the dissolved alendronate.

In the patent with PCT Application No. of WO 01/01991, bisphosphonate tablets with improved surface characteristics have been disclosed.

The patent with PCT Application No. of WO 02/00204 relates to gastric passage of alendronic acid and its salts, and the effect of tannic acid and super dispersants.

The patent with PCT Application No. of WO 98/56360 relates to rapid passage of bisphosphonate tablets through esophagus.

In the patent with PCT Application No. of WO 97/39755, coated dosage forms that contain ibandronate in inner phase to ensure rapid release have been disclosed.

The patent with PCT Application No. of WO 95/00881 contains studies associated with enteric coated alendronate pharmaceutical dosage forms. One of the different aspects of this patent from that study is the use of polymers as coating material that are resistant to gastric acid and are opened at intestinal pH.

Sodium alginate is used in cases of gastrointestinal reflux, heartburn and esophagitis. In the Patent numbered EP-A-0059221, the protective effect of alginic acid and its water soluble salts in gastrointestinal channel have been disclosed. In the patent with PCT Application No. of WO 01/66119, the compositions which cover the sachet formulations of alginic acid and sodium alginate are disclosed and these do not cover the combination of alendronate microparticles with sodium alginate and alginic acid that are coated with polymers resistant to salivary pH. It has been cited that sodium alginate disclosed in the patent with No. of 99/04773, may be used as an ingredient in preparation of pharmaceutical dosage forms but the addition of sodium alginate in therapeutic amounts to the formulation to compensate the esophageal reflux and gastric side effects of alendronate has not been cited. Sodium alginate or alginic acid may be obtained commercially from FMC or Monsanto (for example; Protanal LFR 5/60 or Munucol LB).

### SUMMARY OF THE INVENTION

This invention relates to the pharmaceutical dosage forms that are packaged into sachets, which contain alendronate microparticles coated with a polymer resistant to salivary pH, and the therapeutic amount of alginic acid or sodium alginate and at least one sweetener or a mixture of sweeteners, to be administered orally after dispersed in a glass of 250 ml. water.

Side effects occur with alendronate depending on its irritation of esophagus. To prevent this irritation one of the approaches of this invention is to prevent the contact of alendronate particles with esophagus. To ensure this, alendronate particles are coated with a polymer resistant to salivary pH. Microparticles of alendronate may be prepared by extrusion-rolling, vessel or liquidized bed procedures. The prepared particles are coated with a polymer resistant to salivary pH in a vessel or liquidized bed. For the coating purposes polymers such as aminoalkylmethacrylate copolymers and polyvinyl acetate diethylaminoacetate polymers that are insoluble (neutral pH) in saliva, but soluble in gastric pH may be used. Eudragit E which that is commercially produced in Rohm Pharma can be used in the coating.

The prepared alendronate microparticles are mixed with sodium alginate in a dry environment. After adding the sweetener and the other excipients, the sachet is prepared

To provide a good taste to the formulation, aspartame, potassium acesulfame, sodium saccharine, sucrose and its derivatives, polyols such as mannitol and sorbitol and monoammonium glycyrrhizinate may be used alone or as a mixture.

In the manufacture of the sachets, diluents (i.e., lactose, microcrystalline cellulose) lubricants (i.e., magnesium stearate, talc and PEG 6000), disintegrants (i.e. carboxymethylcellulose, crospovidone, carboxymethyl starch), surfactants, flavors and aromas may be used alone or as mixtures.

To prevent irritant effect of alendronate in mouth and esophagus, alendronate particles are coated with polymers insoluble in neutral pH (neutral pH corresponds to salivary pH). This polymer should be soluble in the gastric pH (pH 1-4) but not in the salivary pH (pH 6-7.5).

The prepared dispersible microparticles sachet formulation when dispersed in a glass of 250 ml. water at the degree of 25° C., alendronate should not be released from the coated alendronate particles preferably in 3 minutes. At the end of 3rd minute, the release of not more 10% w/v of alendronate is permissible.

When with the prepared dispersible microparticles sachet formulation, the dissolution assay is performed in 0.1 N HCl (gastric madium, pH 1.2) at 900 rpm (USP XXIV, paddle method) not less than 85% of alendronate should be dissolved at the end of the 30 minutes.

### EXAMPLE

a. The Manufacture of the Alendronate Microparticles that are Resistant to Salivary pH

Alendronate particle are aggregated with the following mixture:

| | |
|---|---|
| Alendronate | 13.05 mg-91.35 mg |
| PVP K30 | 1 mg-14 mg |
| Ethanol | 6 mg-84 mg |

By the use of the spray coating procedure, alendronate is aggregated with polyvinylprrolidone dissolved in ethanol. The aggregated particles are coated with the following mixture.

| | |
|---|---|
| Eudragit E100 | 10 mg-280 mg |
| Ethanol | 50 mg-400 mg |
| Acetone | 50 mg-400 mg |
| Colloidal silica | 2 mg-15 mg |

b. Preparation of the Sachet

An amount equivalent to 10 mg alendronate acid is taken from the prepared coated microparticles.

Coated microparticles equivalent to 10 mg alendronic acid 26 mg

| | | |
|---|---|---|
| Sucrose | 4648.9 | mg |
| Sodium alginate (Protanal LFR 5/60) | 300 | mg |
| Saccharine sodium | 0.1 | mg |
| PEG 6000 (lubricant) | 25 | mg |

Dissolution Test

The dissolution test has been carried out in 900 ml of 0.1 N HCI (pH 1.2). It has been performed by the use of paddle method under the conditions given in USP XXIV at 50 rpm. At the end of 30 minutes, 89% of alendronate has been dissolved.

Dissolution test of alendronate from the mixture contained in the sachet in salivary pH (neutral pH, pH 6.5):

Since the prepared sachet mixture is to be used after it is dispersed in a glass of water; the mixture is dispersed in a glass of 250 ml. water at the degree of 25° C. and 3 minutes after at pH 6.5 4% of alendronate has been dissolved.

1. A Pharmaceutical formulation, administered orally after dispersing in water at therapeutic doses which comprises of,
a. alendronate microparticles coated with a polymer insoluble at pH 6-7.5, and alginic acid or sodium alginate or admixtures there of, where
b. alendronate dissolves in 900 ml 0.1 N HCl at the rate of not less than 85% of within 30 minutes at the range of pH 1-4,
c. the dispersion in a glass of 250 ml. water at the degree of 25° C. contains no dissolved alendronate at pH 6-7.5 or at the most 10% w/v of alendronate dissolved in 3 minutes.

2. The pharmaceutical formulation as claimed in claim 1, comprises lubricants, diluents, flavors and sweeteners or their mixture thereof.

3. The pharmaceutical formulation as claimed in claim 2, where in the diluent is preferably selected from lactose and microcrystalline cellulose or admixtures thereof.

4. The pharmaceutical formulation as claimed in claim 2, where in the sweetener is selected from, aspartame, potassium acesulfame, monoammonium glycyrrhizinate, sodium saccharine, sucrose and its derivatives, polioles and their derivatives, are preferably used alone or in combination.

5. The pharmaceutical formulation as claimed in claim 1, where in the polymers are selected from, preferably polymethacrylates, polyvinyl acetate diethylaminoacetate and poly butyl methacrylate/2-dimethylamino-ethyl methacrylate/methyl methacrylate copolymers or their mixtures thereof.

6. The pharmaceutical formulation as claimed in claim 1, where in the polymers are, Poly(butyl methacrylate, (2-dimethyl aminoethyl) methacrylate, methyl methacrylate) 1:2:1 is preferred.

7. The pharmaceutical formulation as claimed in claim 1, which is dispersed in a glass of 250 ml. water at the degree of 25° C. at pH 6-7.5, contains alendronate in between 0.001% w/v-3% w/v.

8. The pharmaceutical formulation as claimed in claim 1 where in the alendronate is alendronate monosodium trihydrate or pharmaceutically acceptable derivatives.

9. The pharmaceutical formulation as claimed in claim 1, which is dispersed in a glass of 250 ml. water at the degree of 25° C. at pH 6-7.5, contains alginic acid or sodium alginate or their mixtures in between 0.001% w/v-2% w/v.
